(19) United States
(12) Patent Application Publication
(10) Pub. No.: US 2006/0173358 A1
(43) Pub. Date: Aug. 3, 2006

(54) Fluorescence observation endoscope apparatus and fluorescence observation method

(75) Inventor: Tianyu Xie, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/327,865

(22) Filed: Jan. 9, 2006

(30) Foreign Application Priority Data
Jan. 11, 2005 (JP) 2005-004578
Nov. 4, 2005 (JP) 2005-321314

(51) Int. Cl. A61B 6/00; A61B 1/06

(52) U.S. Cl. 600/476; 600/160

(57) ABSTRACT

A fluorescence observation endoscope apparatus is provided with an endoscope to radiate excitation light from a light source to the inside of a body cavity and pick up a fluorescence figure from a living body tissue in the body cavity; and a fluorescence image generation unit to conduct signal processing of an image pickup signal of the above-described fluorescence figure and generate a fluorescence image, wherein the above-described fluorescence image generation unit includes a target region discrimination unit to calculate the fluorescence level of a predetermined region on the above-described fluorescence image, compare the resulting level with a predetermined fluorescence level, and discriminate a target region on the above-described fluorescence image; and a target region notification unit to notify the presence of the above-described target region.

This application claims benefit of Japanese Application No. 2005-004578 filed in Japan on Jan. 11, 2005 and Japanese Application No. 2005-321314 filed in Japan on Nov. 4, 2005, the contents of which are incorporated by this reference.

### BACKGROUND OF THE INVENTION

1. Field of the Invention

The present invention relates to a fluorescence observation endoscope apparatus and a fluorescence observation method, wherein light is radiated endoscopically, and fluorescence from a living body tissue is observed and diagnosed. In particular, it relates to a fluorescence observation endoscope apparatus and a fluorescence observation method for discriminating a lesion site, e.g., a cancer, and a residue in a body cavity, based on the fluorescence.

2. Description of the Related Art

In recent years, endoscopes capable of optically inspecting the insides of body cavities and, if necessary, conducting treatments by using endo-therapy products have been widely used in the medical field.

The endoscopes have become used also in the case where the endoscopy is conducted based on fluorescence images by using fluorescence other than the case where the endoscopy is conducted in a normal visible region.

For the above-described endoscopic observation based on the fluorescence image, a technology in which autofluorescence from a living body or fluorescence from a drug injected into a living body is detected as a two-dimensional picture, and degeneration of a living body tissue and a status of disease, e.g., a cancer, (for example, type of disease and range of infiltration) are diagnosed from the fluorescence figure thereof is disclosed in U.S. Pat. No. 4,556,057 and U.S. Pat. No. 5,042,494.

When light is radiated to a living body tissue, fluorescence with a wavelength longer than that of the excitation light is emitted. Examples of phosphors in the living body include NADH (nicotinamide adenine nucleotide), FMN (flavin mononucleotide), and pyridine nucleotide. Recently, the correlations between these living-body internal cause substances and diseases are becoming clear.

Furthermore, fluorescent agents, e.g., HpD (hematoporphyrin), Photofrin, and ALA (δ-amino levulinic acid), have a property of accumulating on a cancer and, therefore, disease sites can be diagnosed by injecting these fluorescent agents into living bodies and conducting fluorescence observation.

Technologies for endoscopically diagnosing a lesion site based on the above-described fluorescence include fluorescence observation endoscope apparatuses disclosed in, for example, Japanese Unexamined Patent Application Publication No. 8-224208 and the like.

With respect to the endoscopic observation based on the fluorescence image, for example, fluorescence observation endoscope apparatuses capable of attaining fluorescence images are disclosed in Japanese Unexamined Patent Application Publication No. 7-155285 and Japanese Unexamined Patent Application Publication No. 8-224208.

In the case where the endoscopy based on the fluorescence is conducted, when a fluorescent drug, which tends to accumulate in a lesion site, is administered to a patient or the like and the inside of the body cavity, e.g., the inside of a digestive track, of the patient or the like with a fluorescence endoscope, a residue may exert a significant influence since the residue also emits fluorescence. Therefore, it cannot be determined from the fluorescence image whether the portion emitting the fluorescence in the fluorescence image under observation is due to the fluorescence resulting from the fluorescent drug accumulated in the lesion site or the residue.

Consequently, it is very useful to discriminate and display that the portion emitting the fluorescence in the fluorescence image is due to the fluorescence resulting from the fluorescent drug accumulated in the lesion site or the residue. The fluorescence figure portion due to the residue may interfere the observation and diagnosis based on the fluorescence image.

### SUMMARY OF THE INVENTION

A fluorescence observation endoscope apparatus according to a first aspect of the present invention is provided with an endoscope to radiate excitation light from a light source to the inside of a body cavity and pick up a fluorescence figure from a living body tissue in the body cavity; and a fluorescence image generation unit to conduct signal processing of an image pickup signal of the above-described fluorescence figure and generate a fluorescence image, wherein the above-described fluorescence image generation unit includes a target region discrimination unit to calculate the fluorescence level of a predetermined region on the above-described fluorescence image, compare the resulting level with a predetermined fluorescence level, and discriminate a target region on the above-described fluorescence image; and a target region notification unit to notify the presence of the above-described target region.

A fluorescence observation method according to a second aspect of the present invention includes an image pickup step of picking up an image of fluorescence from a living body tissue in a body cavity by radiating excitation light to the inside of the body cavity; a fluorescence image generation step of conducting signal processing of an image pickup signal of the above-described fluorescence figure and generating a fluorescence image; a target region extraction step of calculating the fluorescence level of a predetermined region on the above-described fluorescence image, comparing the resulting level with a predetermined fluorescence level, and extracting a target region on the above-described fluorescence image; and a target region notification step of notifying the presence of the above-described target region.

### BRIEF DESCRIPTION OF THE DRAWINGS

FIG. 1 is a configuration diagram showing the entire configuration of a fluorescence observation endoscope apparatus to diagnose a lesion site based on fluorescence, according to a first embodiment of the present invention.

FIG. 2 is a characteristic diagram showing the transmission characteristic of an interference filter of a fluorescence filter shown in FIG. 1.

FIG. 3 is a characteristic diagram showing the transmission characteristic of a color filter of the fluorescence filter shown in FIG. 1.

FIG. 4 is a characteristic diagram showing the transmission characteristic of the fluorescence filter and the spectral characteristic of a color image pickup device shown in FIG. 1.

FIG. 5 is a functional block diagram for explaining the functions of an image processing apparatus shown in FIG. 1.

FIG. 6 is a flow chart for explaining the actions of the image processing apparatus shown in FIG. 5.

FIG. 7 is a diagram for explaining the action of the region emergence notification function shown in FIG. 5.

FIG. 8 is a diagram for explaining a first modified example of the action of the region emergence notification function shown in FIG. 5.

FIG. 9 is a diagram for explaining a second modified example of the action of the region emergence notification function shown in FIG. 5.

FIG. 10 is a diagram for explaining a third modified example of the action of the region emergence notification function shown in FIG. 5.

FIG. 11 is a diagram for explaining a fourth modified example of the action of the region emergence notification function shown in FIG. 5.

FIG. 12 is an entire configuration diagram of a fluorescence observation endoscope apparatus according to a second embodiment of the present invention.

FIGS. 13A to 13C are diagrams showing examples of fluorescence spectrum characteristics and fluorescence signal data by a fluorescence filter, attained when excitation light is radiated to lesion sites and the like. FIG. 13A is a diagram showing an example of fluorescence spectrum characteristics based on a fluorescent drug accumulated in a lesion site. FIG. 13B is a diagram showing an example of fluorescence spectrum characteristics based on a residue. FIG. 13C is a diagram showing fluorescence signal data image-captured at different spectra (wavelengths) by a fluorescence filter.

FIG. 14 is a block diagram showing the configuration of an image processing apparatus.

FIG. 15 is a flow chart showing the content of processing by a residue detection method according to the present embodiment.

FIG. 16A is a flow chart showing the content of processing in which a residue is discriminated based on the fluorescence spectrum measurement result. FIG. 16B is a flow chart showing another example of determination processing of steps S25 and S26 shown in FIG. 16A.

FIG. 17 is a diagram showing the configuration in the neighborhood of an image pickup apparatus in the first modified example.

FIG. 18 is a diagram showing the configuration in the neighborhood of an image pickup apparatus in the second modified example.

FIG. 19 is an entire configuration diagram of a fluorescence observation endoscope apparatus according to a third embodiment of the present invention.

FIG. 20 is a diagram showing the spectrum intensity distribution and the like of excitation light radiated through an excitation light filter.

FIG. 21 is a flow chart showing the content of processing, in which a residue is discriminated, in a third embodiment.

FIG. 22 is a flow chart showing the content of processing in which a residue is discriminated.

### DETAILED DESCRIPTION OF THE PREFERRED EMBODIMENTS

The embodiments of the invention will be described with reference to the drawings.

### First Embodiment

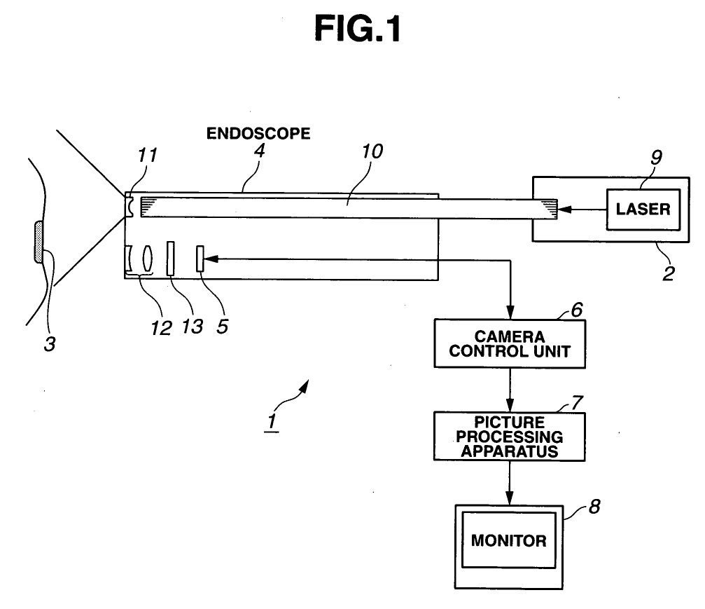

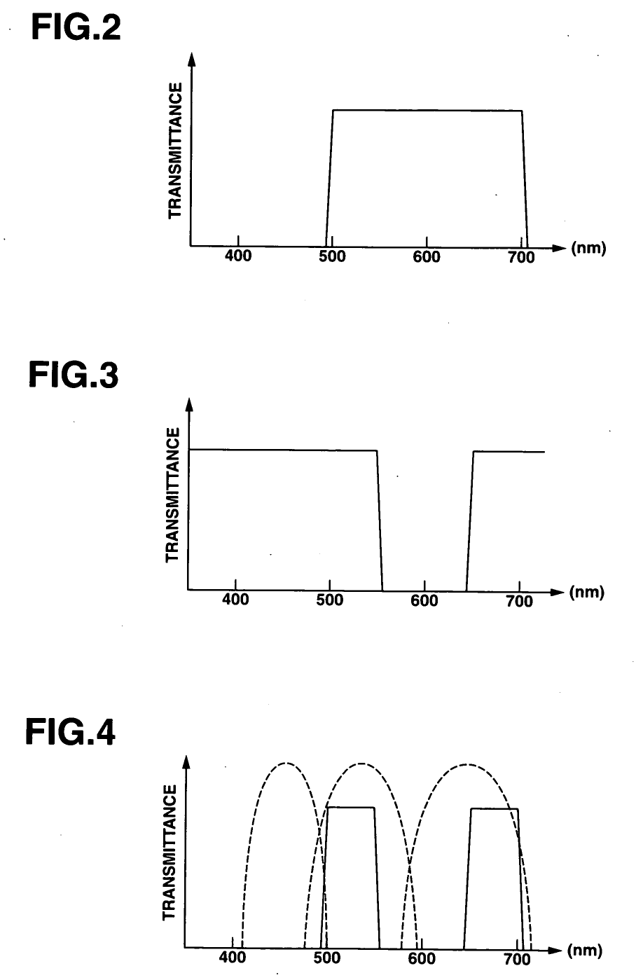

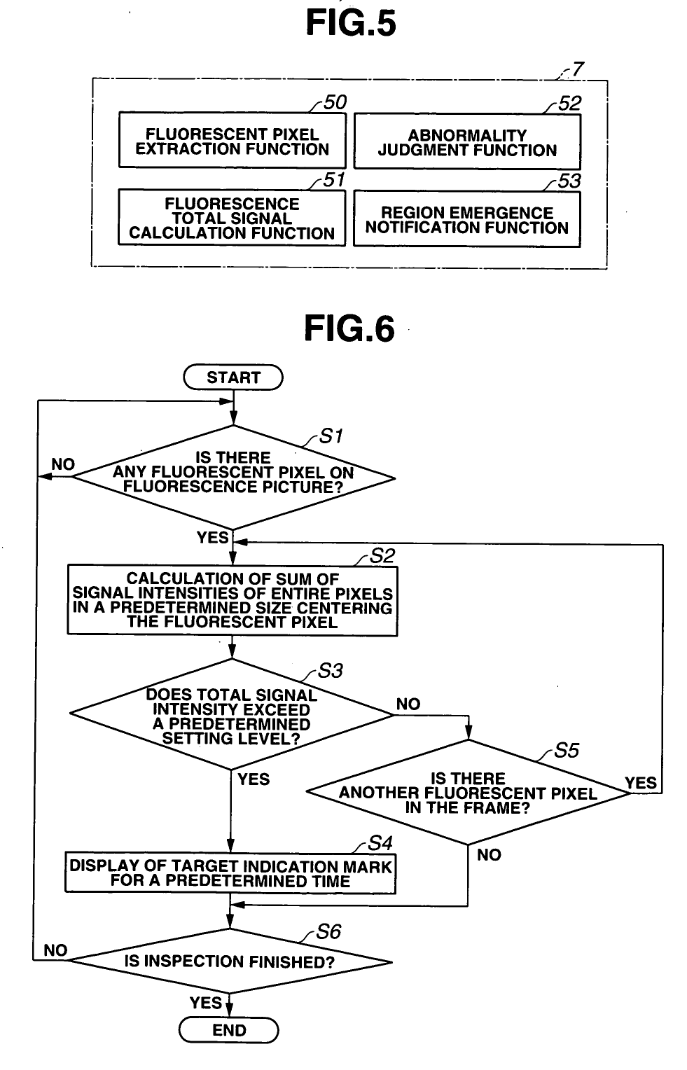

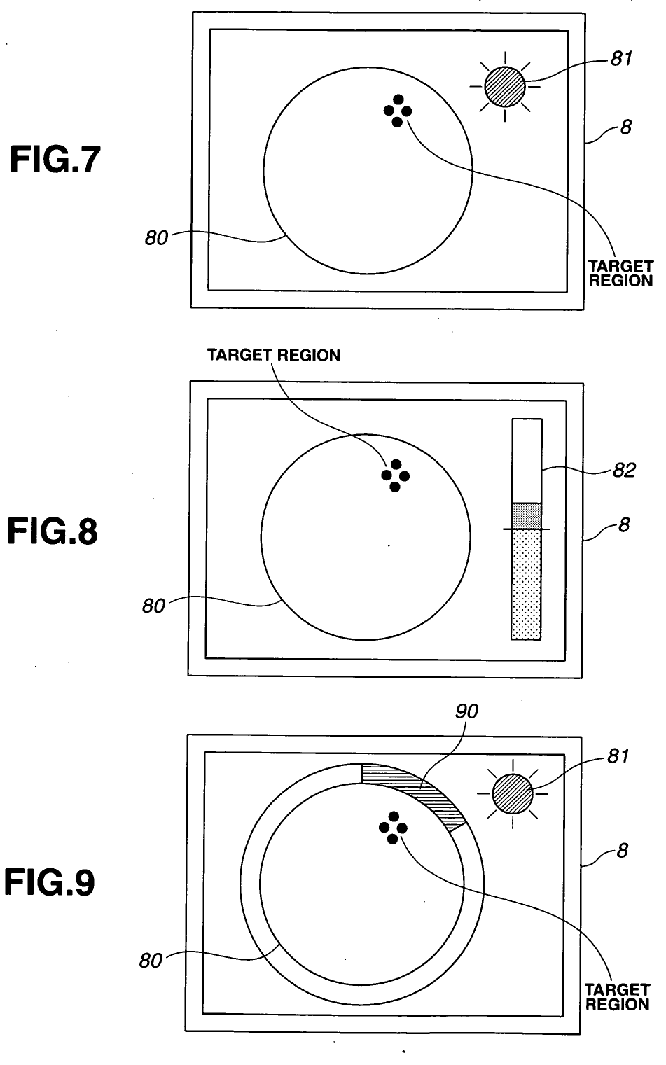

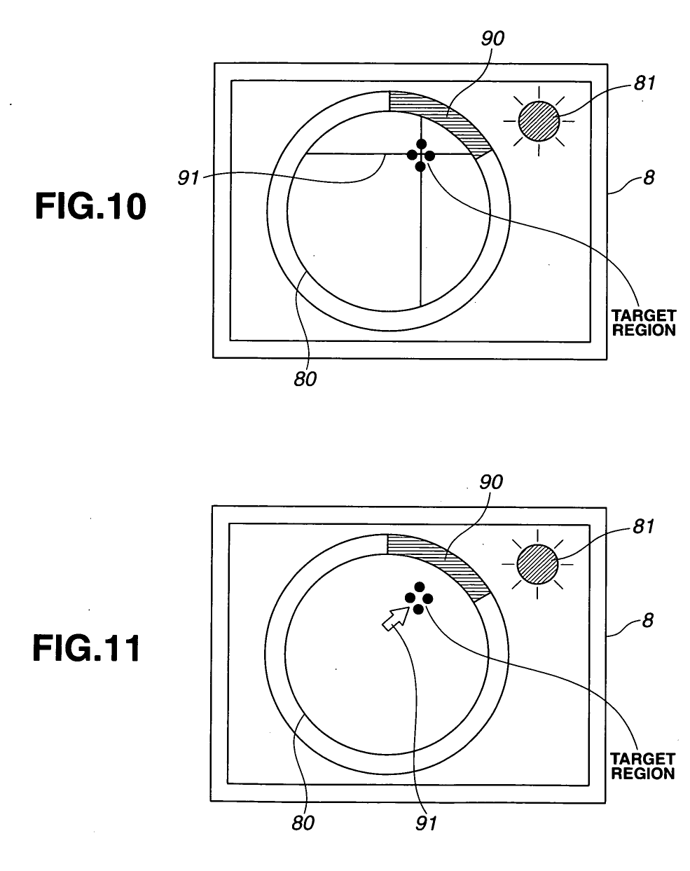

FIG. 1 to FIG. 11 relate to the first embodiment of the present invention. FIG. 1 is a configuration diagram showing the entire configuration of a fluorescence observation endoscope apparatus to diagnose a lesion site based on fluorescence. FIG. 2 is a characteristic diagram showing the transmission characteristic of an interference filter of a fluorescence filter shown in FIG. 1. FIG. 3 is a characteristic diagram showing the transmission characteristic of a color filter of the fluorescence filter shown in FIG. 1. FIG. 4 is a characteristic diagram showing the transmission characteristic of the fluorescence filter and the spectral characteristic of a color image pickup device shown in FIG. 1. FIG. 5 is a functional block diagram for explaining the functions of an image processing apparatus shown in FIG. 1. FIG. 6 is a flow chart for explaining the actions of the image processing apparatus shown in FIG. 5. FIG. 7 is a diagram for explaining the action of the region emergence notification function shown in FIG. 5. FIG. 8 is a diagram for explaining a first modified example of the action of the region emergence notification function shown in FIG. 5. FIG. 9 is a diagram for explaining a second modified example of the action of the region emergence notification function shown in FIG. 5. FIG. 10 is a diagram for explaining a third modified example of the action of the region emergence notification function shown in FIG. 5. FIG. 11 is a diagram for explaining a fourth modified example of the action of the region emergence notification function shown in FIG. 5.

As shown in FIG. 1, a fluorescence observation endoscope apparatus 1 of the present embodiment comprises a light source 2 to emit light (excitation light) in a blue or ultraviolet region, an endoscope 4 to lead the excitation light into a living-body cavity and observe fluorescence emitted from a lesion site 3, a camera control unit 6 to drive a color image pickup device 5 built in the endoscope 4 and convert a fluorescence figure of the lesion site 3 to a video signal, an image processing apparatus 7 to process the video signal so as to facilitate discrimination between the lesion site 3 and a normal portion, and a monitor 8 to display the output from the image processing apparatus 7 as an image. The image processing apparatus 7 is provided with a fluorescence image generation unit to generate a fluorescence signal data.

The above-described light source 2 incorporates a laser 9, e.g., excimer, He—Cd, or argon, to emit light in a blue or ultraviolet region. The above-described endoscope 4 is configured to include a light guide 10 to lead laser light emitted from the above-described laser 9 to the living-body cavity, a concave lens 11 to diffuse the laser light for illumination, objective lenses 12 to project a fluorescence figure of the lesion site 3 to the color image pickup device 5, and a fluorescence filter 13 to transmit specific wavelengths of the fluorescence figure from the objective lenses 12.

The endoscope 4 in the fluorescence observation endoscope apparatus 1 is configured to include a light guide 10 to lead laser light emitted from the laser 9 to the living-body cavity, the concave lens 11 to diffuse the laser light for illumination, the objective lenses 12 to project a fluorescence figure of the lesion site 3 to the color image pickup device 5, and the fluorescence filter 13 to transmit specific wavelengths of the above-described fluorescence figure. The fluorescence filter 13 has transmission characteristics of transmitting green of 500 nm to 540 nm and red of 640 nm to 700 nm.

The fluorescence filter 13 comprises an interference filter having a transmission characteristic shown in FIG. 2 and a color filter having a transmission characteristic shown in FIG. 3. As a result, the fluorescence filter 13 has the transmission characteristics indicated by solid lines in FIG. 4.

Although not shown in the drawing, the above-described light source 2 includes a xenon lamp for emitting white light and a switching unit to switch between the laser 9 and the xenon lamp and supply to the light guide 10. Furthermore, the endoscope 4 incorporates an image pickup device, although not shown in the drawing, to pick up an image by the white light.

The image processing apparatus 7 serving as a fluorescence image generation unit includes a fluorescent pixel extraction function 50, a fluorescence total signal calculation function 51, an abnormality determination function 52, and a region emergence notification function 53, as shown in FIG. 5. Each of these functions will be described below in detail.

In the thus configured image processing apparatus 7 of the present embodiment, as shown in FIG. 6, the fluorescent pixel extraction function 50 scans one frame of fluorescence image image-captured with the color image pickup device 5 and extract fluorescent pixels on the fluorescence image in step S1.

Here, the fluorescent pixel refers to a pixel exhibiting a luminance exceeding the background level of one frame of fluorescence image by a predetermined level, for example, 20%.

When a fluorescent pixel is detected, the fluorescence total signal calculation function 51 sums signal intensities of all pixels in a region of a predetermined size centering the fluorescent pixel and, therefore, the total signal intensity is calculated in step S2.

Subsequently, the abnormality determination function 52 serving as a target region discrimination unit determines whether the fluorescence is originated from an abnormal tissue, e.g., a cancer, which is a target region, based on whether the resulting total signal intensity exceeds a predetermined set level or not in step S3.

When the total signal intensity exceeds the predetermined set level and the fluorescence is determined as being originated from an abnormal tissue, e.g., a cancer, the region emergence notification function 53 displays a region indication mark described below for a predetermined time on a monitor 8 in step S4. Thereafter, shift to a frame is conducted, followed by step S6.

When the total signal intensity is lower than the predetermined set level, it is determined that the fluorescence is not originated from an abnormal tissue, e.g., a cancer, and in step S5, the fluorescence image is scanned to check whether another fluorescent pixel is present in the same frame. When another fluorescent pixel is present, the sequence returns to step S2, and when not present, shift to a frame is conducted, followed by step S6.

In step S6, it is determined whether there is an instruction to finish the inspection with the fluorescence observation endoscope apparatus 1. When there is no instruction to finish the inspection, the process returns to step 1, and when there is an instruction to finish the inspection, the process is finished.

Here, the processing for displaying the region indication mark by the region emergence notification function 53 serving as a target region notification unit in step S4 will be described.

As shown in FIG. 7, when the total signal intensity exceeds the predetermined set level and the fluorescence is determined as being originated from an abnormal tissue, e.g., a cancer, which is a target region, the region emergence notification function 53 of the image processing apparatus 7 allows a region indication mark 81 to be displayed at a position adjacent to a fluorescence image 80 displayed on the monitor 8 and, thereby, the presence of the abnormal tissue, e.g., a cancer, is notified. At this time, the luminance of the region indication mark 81 is allowed to reflect the size of the abnormal tissue, e.g., a cancer.

In this manner, in the present embodiment, the region indication mark 81 is displayed on the monitor 8 when a fluorescence region in a certain region on the fluorescence screen exceeds a predetermined set level. Therefore, oversight of the presence of a lesion site can be reliably prevented even when the lesion site or the like is small and the fluorescence intensity is low.

A speaker, although not shown in the drawing, may be disposed in the image processing apparatus 7 and the region emergence notification function 53 may notify the presence of the abnormal tissue, e.g., a cancer, by a sound using the speaker.

Alternatively, as shown in FIG. 8, the region emergence notification function 53 may notify the presence of the abnormal tissue, e.g., a cancer, by displaying a region indicator 82 at a position adjacent to the fluorescence image 80 displayed on the monitor 8 when the total signal intensity exceeds the predetermined set level and the fluorescence is determined as being originated from the abnormal tissue, e.g., a cancer. This region indicator 82 can conduct notification reflecting the size and the like of a target region, a lesion site, by displaying the intensity of the fluorescence while changing the level or the color.

Further, in the region emergence notification function 53, as shown in FIG. 9, a direction navigation display portion 90 is disposed in the perimeter portion of the fluorescence image 80 in addition to the region indication mark 81. In this case, when the total signal intensity exceeds the predetermined set level and the fluorescence is determined as being originated from the abnormal tissue, e.g., a cancer, a part of the direction navigation display portion 90 in the direction of the presence of the abnormal tissue, e.g., a cancer, is lit and, thereby, the notification is conducted.

Furthermore, as shown in FIG. 10 and FIG. 11, the region emergence notification function 53 may display a position navigation display portion 91 at a position in the vicinity of or adjacent to the target region in the inside of the fluorescence image 80.

FIG. 10 shows an example in which the position navigation display portion 91 is displayed by a cross hair, and it is indicated that the position of the target region is in the neighborhood of the intersection point of the cross hair. FIG. 11 shows an example in which the position navigation display portion 91 is displayed by an arrow (either light up or flash), and it is indicated that the position of the target region is in the neighborhood of the front end of the arrow.

In the present embodiment, if the sum of luminance of the entire pixels of the fluorescence image is lower than or equal to the predetermined level, the extraction of fluorescent pixel can be stopped. This is because it is believed that each pixel has a large noise component in such a case and there is a doubt about the reliability in measurement.

According to the first embodiment of the present invention, a fluorescence observation endoscope apparatus capable of reliably notifying the presence of a lesion site even when the lesion site or the like is small and the fluorescence intensity is low can be realized.

In the fluorescence observation endoscope apparatus 1 described in the above-described embodiment, a function of determining whether the portion emitting fluorescence in the fluorescence image under observation is the fluorescent drug accumulated in a lesion site or a residue may be provided since the residue also emits fluorescence when, for example, the inside of digestive track is inspected. The following embodiment can be adopted as an example in which the fluorescence observation endoscope apparatus 1 has the function of discriminating a fluorescence figure portion of a residue, as described above, when the inside of a body cavity is subjected to fluorescence image pickup.

### Second Embodiment

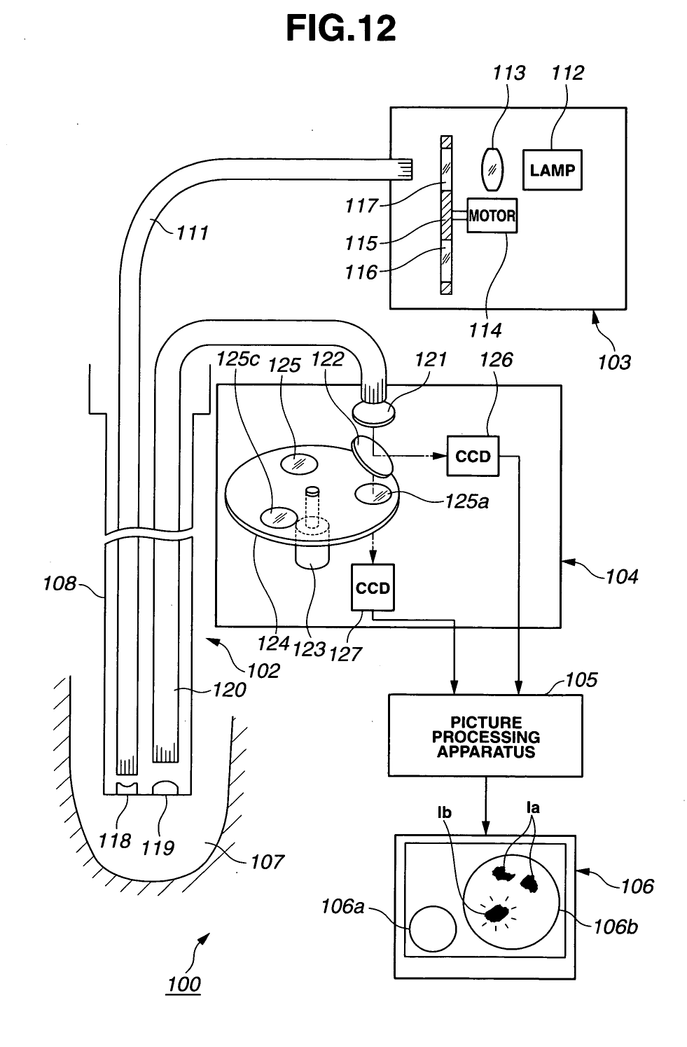

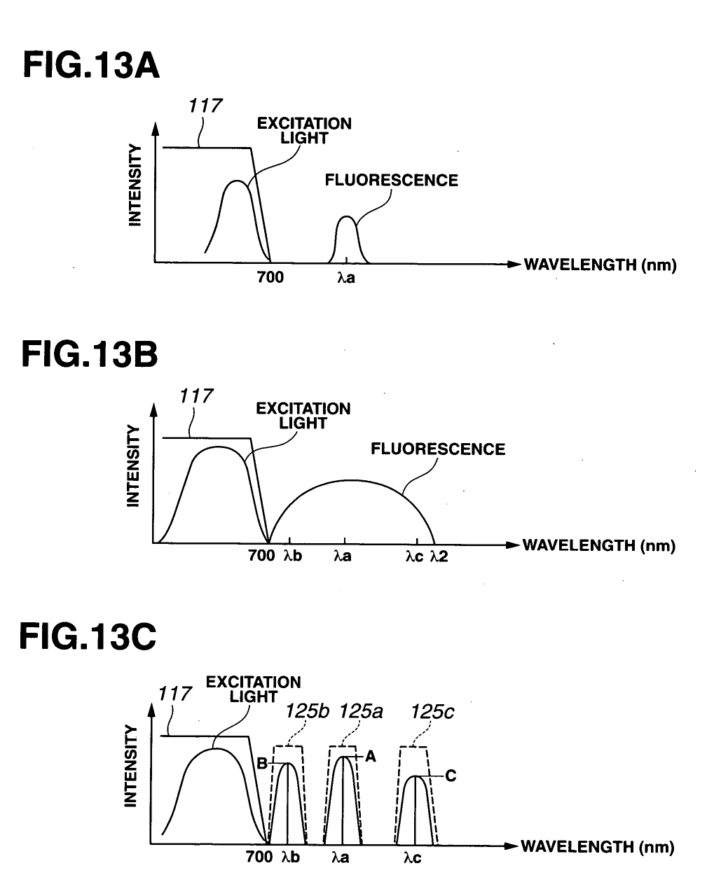

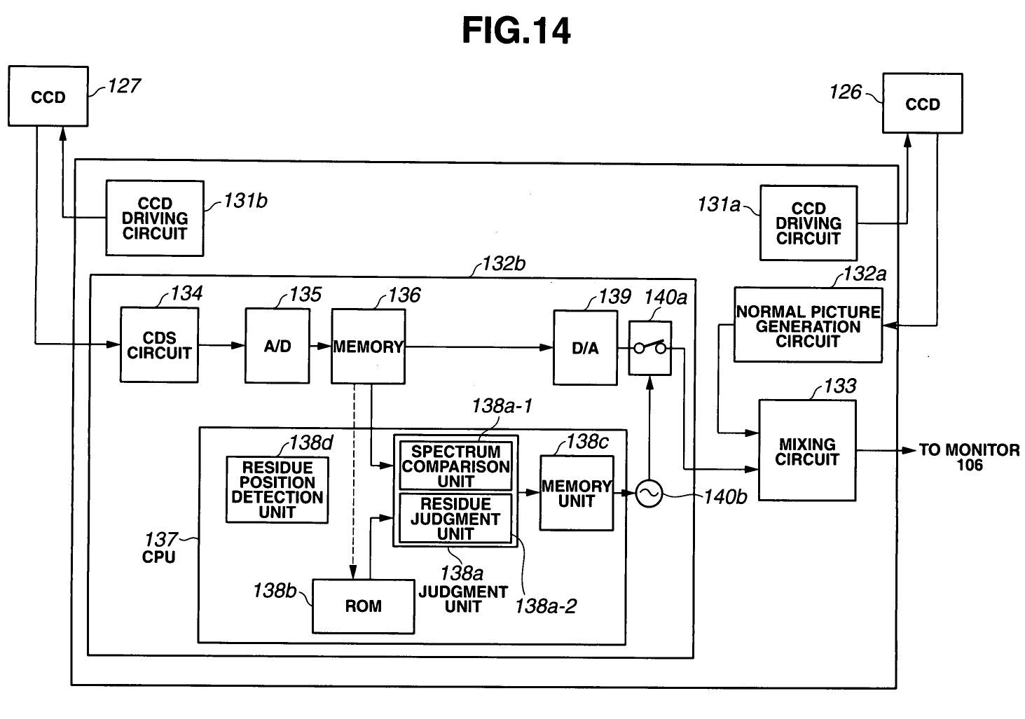

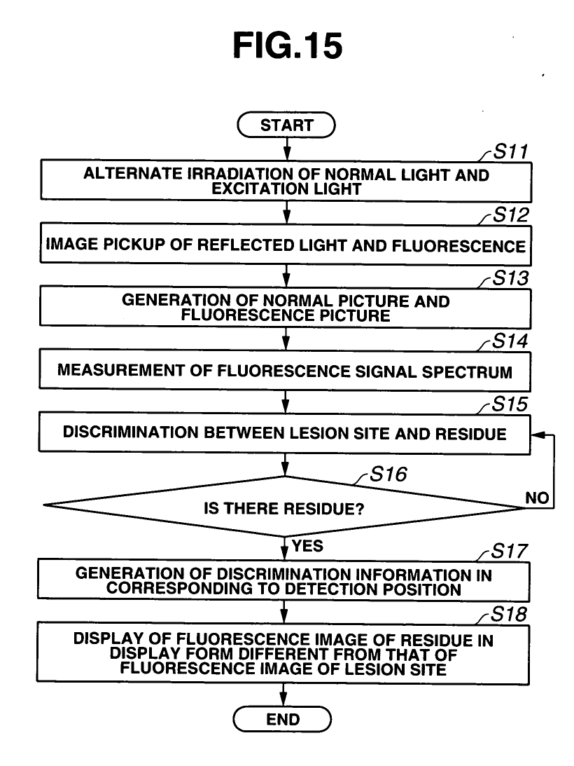

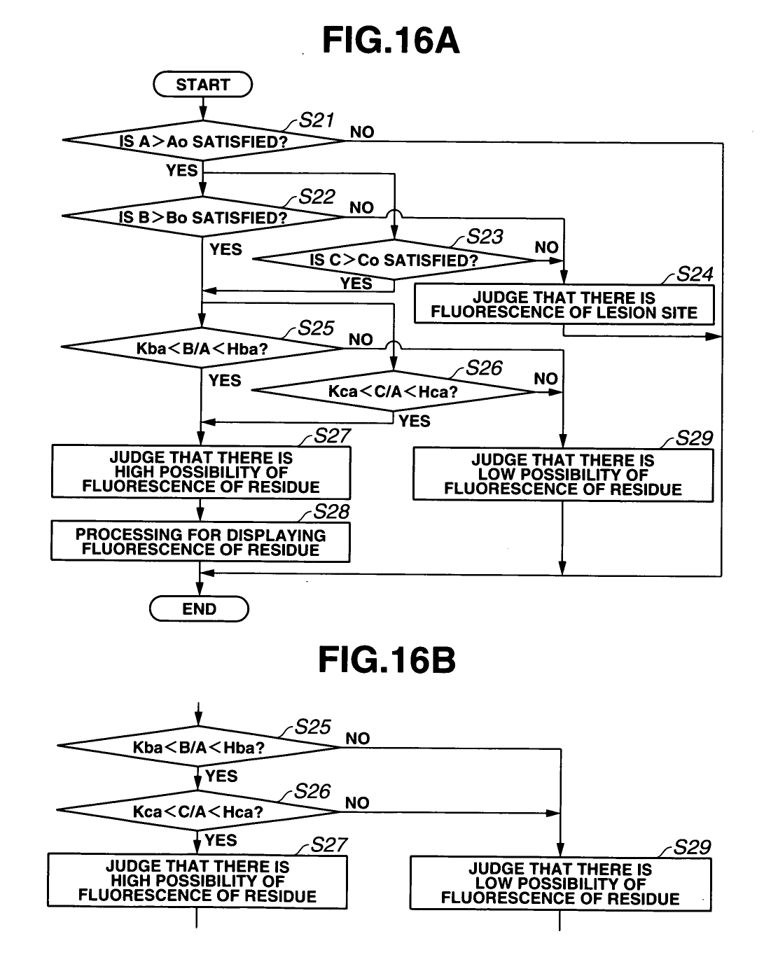

FIG. 12 to FIG. 16 relate to the second embodiment of the present invention. FIG. 12 shows the configuration of a fluorescence observation endoscope apparatus according to the second embodiment of the present invention. FIG. 13A to FIG. 13C show examples of fluorescence spectrum characteristics attained when excitation light is radiated to lesion sites and the like and fluorescence signal data by a fluorescence filter. FIG. 14 shows the configuration of an image processing apparatus. FIG. 15 shows the content of processing by a residue detection method according to the present embodiment. FIG. 16A shows the content of processing in which a residue is discriminated based on the fluorescence spectrum measurement result. FIG. 16B shows another example of a determination processing of steps S25 and S26 shown in FIG. 16A.

As shown in FIG. 12, a fluorescence observation endoscope apparatus 100 in the second embodiment of the present invention comprises an optical endoscope 102 to be inserted into a body cavity 107, a light source 103 to supply excitation light and illumination light to a light guide 111 to transmit the illumination light of this endoscope 102, an image pickup apparatus 104 to pick up a normal image by white light (light in a visible region) and a fluorescence image with respect to an optical image attained by receiving the light with the endoscope 102, an image processing apparatus (including a fluorescence image generation unit) 105 to conduct image processing of image pickup signal image-captured by this image pickup apparatus 104, and a monitor 106 to display an image processed by this image processing apparatus 105.

The endoscope 102 includes an insertion portion 108 to be inserted into the body cavity 107. The light guide 111 to transmit the illumination light is inserted into this insertion portion 108, and the rear end of this light guide 111 is connected to the light source 103.

A lamp 112 to emit, for example, white light as a light source to emit illumination light is built in the light source 103. The illumination light emitted from this lamp 112 is condensed by a condenser lens 113, passes a white-light-transmitting filter (abbreviated as a white-light filter) 116, which is attached to a rotary plate 115 driven to rotate by a motor 114 and transmits white light, and an excitation-light-transmitting filter (hereafter abbreviated as an excitation light filter) 117 alternately, and is incident on an incident end surface at the rear end of the light guide 111.

The light incident on the incident end surface of the light guide 111 is transmitted to the front-end surface thereof, and is radiated to the side of a subject, e.g., a lesion site or the like, in the body cavity 107 from the front-end surface through an illumination lens 118 attached to an illumination window while being diverged. That is, an excitation light irradiation unit to radiate the excitation light in one band to the inside of the body cavity is composed of the lamp 112 and the excitation light filter 117.

An observation window is disposed adjacent to the illumination window at the distal end portion of the insertion portion 108. An objective lens 119 is attached to this observation window, and an optical image of the subject is generated at the image generation position of the objective lens 119. A front-end surface of an image guide 120 is disposed at this image generation position. The optical image generated on the front-end surface of the image guide 120 is transmitted to an end surface on the proximal side, and is image-captured by the image pickup apparatus 104 attached to the end surface.

That is, an image pickup lens 121 is disposed facing the rear-end surface of the image guide 120, and on an optical axis thereof, a dichroic mirror 122 and a rotary plate 124 rotated by a motor 123 are disposed. Here, the dichroic mirror 122 divides the light incident from the inside of the body cavity 107 on the observation window of the insertion portion 108 into two types, normal light and fluorescence. Furthermore, fluorescence filters 125a, 125b, and 125c, which are set in such a way that the excitation light is cut and the fluorescence in a plurality of different wavelength bands is transmitted, are attached to this rotary plate 124.

The light emitted from the rear-end surface of the image guide 120 is condensed by an image pickup lens 121. At that time, an image of the light reflected by the dichroic mirror 122 is generated on an image pickup surface (light-receiving surface) of a charge-coupled device (abbreviated as CCD) 126 disposed on an optical axis of the reflected light and serving as a solid-state image pickup device for normal observation, and a normal image is image-captured by this CCD 126.

On the other hand, the light passed this dichroic mirror 122 is passed through the fluorescence filters 125a, 125b, and 125c disposed sequentially on an optical axis, and an image thereof is generated on an image pickup surface of a CCD 127 for observing fluorescence, disposed at an image generation position on the optical axis of the image pickup lens 121. That is, a fluorescence image pickup unit is composed of the dichroic mirror 122 and the CCD 127.

The signal subjected to photoelectric conversion by both the CCD 126 and CCD 127 is input into the image processing apparatus 105, and is subjected to image processing. Thereafter, the images image-captured by the CCD 126 and CCD 127 are displayed on the monitor 106.

The fluorescence filters 125a, 125b, and 125c attached to the above-described rotary plate 124 are set in correspondence to the wavelength of fluorescence intrinsically (or specifically) emitted by a fluorescent drug administered to an affected area or the like and the wavelength of fluorescence intrinsically (or specifically) emitted by a residue.

This will be specifically described. The fluorescent drug administered to an affected area has an intrinsic spectrum characteristic in which fluorescence exhibiting a peak at a wavelength of λa is emitted relative to the excitation light as shown in FIG. 13A.

In the present embodiment and the like, it is primarily described that when lesion (or a lesion site) is present in an affected area, a fluorescent drug having a characteristic of accumulating on the site is administered and, thereby, a fluorescence figure of the lesion site is attained based on an observation of fluorescence emitted from the fluorescent drug. However, the same can hold true for the case where a lesion site itself exhibits autofluorescence even when no fluorescent drug is administered. Therefore, in order to include these cases, a simplified expression, fluorescence of lesion site, is also used in the present specification and Claims, even when it is appropriate to express as fluorescence originated from a fluorescent drug accumulated on a lesion site.

In contrast to the above-described FIG. 13A, a residue has an intrinsic spectrum characteristic in which fluorescence is emitted over a relatively broad wavelength band from a wavelength of 700 nm to a longer wavelength of λ2, as shown in FIG. 13B.

That is, for the lesion site, fluorescence is emitted with a relatively narrow wavelength range in the neighborhood of λa. On the other hand, for the residue, a wavelength of λb shorter than λa and a wavelength of λc longer than λa are included in addition to this wavelength of λa.

Consequently, in the present embodiment, a fluorescence spectrum is generated while center wavelengths of transmission bands are set at the wavelength of λa and wavelengths of both sides thereof, λb and λc, as shown in FIG. 13C (each of them is indicated by a broken line), for the transmission wavelengths of the fluorescence filters 125a, 125b, and 125c with respect to band-pass filter characteristics. That is, a spectrum generation unit is composed of a plurality of fluorescence filters 125a, 125b, and 125c in which transmission in a plurality of transmission bands is effected, and the above-described fluorescence signal spectrum is generated based on the fluorescence which have passed the plurality of bands.

In this case, the filter characteristics are set targeting the wavelength band of the fluorescence emitted, that is, light with a wavelength of near-infrared or longer. Therefore, based on the measurement results of the thus attained spectrum, it is possible to discriminate that the fluorescence detected only in the case of the fluorescence filter 125a with a wavelength of λa is originated from a lesion site and the fluorescence detected also in the case of fluorescence filters 125b and 125c with wavelengths of λb and λc on both sides in addition to the case of the fluorescence filter 125a with a wavelength of λa is originated from a residue.

FIG. 14 shows the configuration for conducting image processing in the image processing apparatus 105.

The CCD 126 is driven by a CCD driving signal being applied from a CCD driving circuit 131a, and an image pickup signal corresponding to the white image subjected to photoelectric conversion in the CCD 126 is input into a normal image generation circuit 132a. The image pickup signal input into this normal image generation circuit 132a is converted to a video signal and, thereafter, is input into a mixing circuit 133. In this mixing circuit 133, the video signal corresponding to the white image (normal image) image-captured in the CCD 126 and the video signal corresponding to the fluorescence image image-captured in the CCD 127 are mixed and output on the monitor 106, and both images are displayed on the monitor 106.

The CCD 127 is driven by a CCD driving signal being applied from a CCD driving circuit 131b, and an image pickup signal of fluorescence corresponding to the fluorescence image subjected to photoelectric conversion in the CCD 127 is input into a CDS circuit 134 constituting a fluorescence image generation circuit 132b.

After a signal component is extracted by this CDS circuit 134, the signal component is converted to a digital signal by an A/D converter 135, and is stored into a memory 136.

The fluorescence signal data stored in this memory 136 is read by CPU 137 serving as a residue detection unit to conduct processing for discriminating whether each portion in the fluorescence signal data is originated from the lesion site or originated from the residue. The comparison with a threshold value stored beforehand in, for example, a ROM 138b is conducted by a determination unit 138a in the CPU 137, and the determination result thereof is stored in, for example, a memory unit 138c. The determination unit 138a is provided with a spectrum comparison unit 138a-1 to compare the fluorescence signal data with the threshold value stored beforehand in the ROM 138b and a residue determination unit 138a-2 to determine the presence or absence of residue based on the information attained in this spectrum comparison unit 138a1.

The fluorescence signal data of each frame image-captured after sequentially passing through the fluorescence filters 125a, 125b, and 125c shown in FIG. 12 is stored in the memory 136 in such a way that the data of three frames are linked together as one set.

In the determination unit 138a, each of the fluorescence signal data as measurement data image-captured by the three fluorescence filters 125a, 125b, and 125c shown in FIG. 13C, is compared with the threshold value stored beforehand in the ROM 138b, and based on the comparison results, the information (for example, flag code) for discriminating between the fluorescence signal data of the lesion site and the fluorescence signal data of the residue is stored in the memory unit 138c. An intrinsic fluorescence spectrum of the lesion site in the above-described body cavity or a residue fluorescence spectrum is used as the threshold value stored beforehand in the ROM 138b.

In the memory unit 138c, the discrimination information is stored at the same address as, for example, that in the memory 136. When the fluorescence signal data stored in the memory 136 is read and displayed on the monitor 106, the address for reading, applied to the memory 136, is also applied to the memory unit 138c, and the corresponding discrimination information is read synchronously.

Consequently, the fluorescence figure originated from the residue, displayed on the monitor 106, is displayed in a display form different from that of the fluorescence figure of the lesion site, based on the discrimination information in correspondence to the position and the size of the residue by using CPU 137. As described above, the CPU 137 is provided with a residue position detection unit 138d to detect the position and the size of a residue.

The determination by the determination unit 138a can be conducted on a pixel basis. However, an identification code may not be assigned to a fluorescence figure of a residue having an adequately small size in the vision. When a small point-like size is also displayed in a display form different from the fluorescence figure of the lesion site, the display form may become an obstruction. Therefore, the dimension of the pixel size may be included in the criteria of determination (it is preferable that the pixel size can be selected and set on the user side).

The ROM 138b is formed from, for example, EEPROM, flash memory, or the like serving as non-volatile memory capable of being electrically rewritten. For the purpose of determining a residue, it can also be made possible that a portion which is known (ascertained) beforehand as a residue is image-captured, the fluorescence signal data in that case is stored in the memory 136, and the CPU 137 is controlled by the instruction operation, although not shown in the drawing, in such a way as to store the fluorescence signal data in the ROM 138b (as indicated by a broken line shown in FIG. 14), and if necessary, correct and set the fluorescence signal data as a threshold value.

The fluorescence signal data image-captured through the fluorescence filter 125a in the above-described memory 136 is converted to an analog video signal through a D/A converter 139, input into the mixing circuit 133, and is mixed with the video signal on the CCD 126 side so as to be output to the monitor 106.

In this case, for example, an analog switch 140a serving as a residue figure processing unit is disposed on the way from the D/A converter 139 to the input into the mixing circuit 133. An oscillator 140b is disposed in between the analog switch 140a and the memory unit 138c. By this configuration, the oscillation of the oscillator 140b is controlled based on the information on the determination result from the memory unit 138c and ON/OFF of the analog switch 140a is conducted.

That is, when the fluorescence is determined as being originated from the lesion site, as described below, the analog switch 140a remains ON. However, when the discrimination signal relating to the determination indicating that there is a high possibility of residue is stored in the memory unit 138c, the analog switch 140a is turned ON/OFF by the oscillation output of the oscillator 140b. The fluorescence image portion, which has been determined as being a residue with a high possibility, is displayed while flashing.

The operations according to the present embodiment will be described below. As shown in FIG. 12, the end portion on the proximal side of the light guide 111 of the endoscope 102 is connected to the light source 103, and the rear end of the image guide 120 is connected to the image pickup apparatus 104.

The power of the light source 103 and the like is turned on, and the insertion portion of the endoscope 102 is inserted into the body cavity 107. As indicated by step S11 shown in FIG. 15, the illumination light from the lamp 112 of the light source 103 is radiated to the living body tissue side in the body cavity 107 through the light guide 111, the front-end surface thereof, and the illumination lens 118.

In this case, since the white light filter 116 and the excitation light filter 117 attached to the rotary plate 115 rotated by the motor 114 are disposed alternately on an illumination optical path of the lamp 112, the white light (normal light) and the excitation light are radiated to the living body tissue alternately.

A part of the white light and the excitation light radiated to the living body tissue become reflected light, and an image thereof is generated on the front-end surface of the image guide 120 by the objective lens 119. Furthermore, an image of the fluorescence emitted by the excitation light is also generated on the front-end surface of the image guide 120 by the objective lens 119. Subsequently, these are emitted from the rear-end surface of the image guide 120 to the image pickup apparatus 104 side.

As indicated by step S12 shown in FIG. 15, image pickup (light reception) is conducted by the CCD 126 for normal observation and the CCD 127 for fluorescence observation.

That is, the light emitted from the rear-end surface of the image guide 120 is condensed by the image pickup lens 121. At that time, for the white light, the wavelength component substantially in the visible region except for the fluorescence components on the side of wavelengths longer than about 700 nm shown in FIG. 13 is reflected by the dichroic mirror 122, and an image is generated on the image pickup surface (light reception surface) of the CCD 126 for normal observation, disposed on the optical axis of the reflected light, so that a normal image (white image) is image-captured by this CCD 126.

On the other hand, in the case where the excitation light passed through the excitation light filter 117 is radiated, the excitation light components on the short wavelength side are cut by the dichroic mirror 122 and, in addition, only the fluorescence components on the side of wavelengths longer than about 700 nm are passed through this dichroic mirror 122. Subsequently, images are picked up by the CCD 127 for fluorescence observation through the fluorescence filters 125a, 125b, and 125c sequentially disposed on the optical axis.

The signals image-captured by these CCD 126 and CCD 127 are transferred to the image processing apparatus 105, and video signals corresponding to the normal image and the fluorescence image, respectively, are generated, as indicated by step S13 shown in FIG. 15.

In a display example on the monitor 106 shown in FIG. 12, the normal image image-captured by the CCD 126 is displayed after being subjected to a reduction treatment.

On the other hand, for the fluorescence image image-captured by the CCD 127, a spectrum measurement processing of the fluorescence signal is conducted, as indicated by step S14 shown in FIG. 15. Furthermore, as indicated by step S15, processing for determining (discriminating) whether the fluorescence figure is originated from a lesion site or the fluorescence figure is originated from a residue is conducted based on the results of the spectrum measurement processing.

As indicated by step S16, processing for detecting the presence or absence of residue based on this determination processing is conducted repeatedly with respect to the signal data of each frame displayed on the monitor 106.

That is, the processing for determining whether the fluorescence figure is originated from a lesion site or the fluorescence figure is originated from a residue is conducted by the determination unit 138a shown in FIG. 14. For this determination processing, the fluorescence signal data stored in the memory 136 are read sequentially, and are compared with the threshold value on a pixel basis, so as to conduct the determination. Subsequently, the discrimination information in correspondence to the determination result is stored in the memory unit 138c. That is, as indicated by step S17 shown in FIG. 15, the discrimination information in correspondence to the position of each pixel subjected to the determination processing is generated.

As indicated by step S18, in the case where the fluorescence figure originating from the lesion site and the fluorescence figure originating from the residue are displayed based on the result of the above-described determination processing, the fluorescence figure of the residue is displayed on the monitor 106 in a display form different from that of the fluorescence figure of the lesion site, for example, the fluorescence figure of the residue is displayed while flashing.

As described above, by displaying the fluorescence figure of the residue on the monitor 106 in the display form different from that of the fluorescence figure of the lesion site, even in the case where the fluorescence figure of the residue is displayed while being mixed with the fluorescence figure of the lesion site, the user can discriminate visually the fluorescence figure of the residue from the fluorescence figure of other fluorescence figures (fluorescence figure of the lesion site in the present embodiment) easily. Therefore, diagnosis and the like can be conducted efficiently.

The processing of steps S15 and S16 shown in FIG. 15, that is, the content of the determination processing by the determination unit 138a shown in FIG. 14 will be described below in more detail with reference to FIG. 16.

As is described for the configuration of the CPU 137 shown in FIG. 14, the fluorescence signal data image-captured sequentially with spectra (wavelengths) different depending on the fluorescence filters 125a, 125b, and 125c are stored in the memory 136.

In the determination unit 138a, the signal data image-captured through the fluorescence filter 125a is compared with a threshold value Ao stored in the ROM 138b, the signal data image-captured through the fluorescence filter 125b is compared with a threshold value Bo stored in the ROM 138b, and the signal data image-captured through the fluorescence filter 125c is compared with a threshold value Co stored in the ROM 138b.

In the following description, the signal data image-captured through the fluorescence filters 125a, 125b, and 125c are represented by A, B, and C, respectively, as shown in FIG. 13C.

That is, the determination unit 138b of the CPU 137 determines whether the values of the image-captured fluorescence signal data A, B, and C exceed the threshold values Ao, Bo, and Co or not, as indicated by steps S21, S22, and S23 shown in FIG. 16A.

In step S21, when the fluorescence signal data A is larger than the threshold value Ao, the determination unit 138a determines whether the fluorescence signal data B is larger than the threshold value Bo or not and whether the fluorescence signal data C is larger than the threshold value Co or not, as indicated by steps S22 and S23. Conversely, when the fluorescence signal data A larger than the threshold value Ao is not detected in step S21, it is determined that there is almost no fluorescence of lesion site nor fluorescence of residue, and this processing is finished.

In the determination processing of steps S22 and S23, when the signal data B and C do not exceed the threshold values Bo and Co, respectively, the process proceeds to step S24, wherein the determination unit 138a determines that the signal data A in this case is not the fluorescence of residue, but is the fluorescence of lesion site, and this processing is finished.

On the other hand, in the determination processing of steps S22 and S23, when the signal data B and C exceed the threshold values Bo and Co, since the fluorescence which may be originated from a residue is detected, the process proceeds to the determination processing of steps S25 and S26.

In step S25, the determination unit 138a conducts comparison determination whether the signal data ratio B/A is in between the two threshold values Kba and Hba or not, which are the value based on the envelope of the residue fluorescence signal spectrum in the neighborhood of a wavelength λb and the value in the neighborhood of a wavelength λa and are used for determining the range corresponding to a residue with a high possibility. The threshold value Kba is a lower limit side threshold value and Hba is an upper limit side threshold value.

Likewise, in step S26 as well, the determination unit 138a conducts comparison determination whether the signal data ratio C/A is in between the two threshold values Kba and Hba, which are the value based on the envelope of the residue fluorescence signal spectrum in the neighborhood of a wavelength λc and the value in the neighborhood of a wavelength λa and are used for determining the range corresponding to a residue with a high possibility. The threshold value Kca is a lower limit side threshold value and Hca is an upper limit side threshold value.

When the condition Kba<B/A<Hba is satisfied or the condition Kca<C/A<Hca is satisfied in the determination processing of steps S25 and S26, step S27 is executed. In this case, the determination unit 138a determines that there is a high possibility of residue. As is described in FIG. 14, the determination unit 138a writes the discrimination information based on the determination result into the memory unit 138c. This discrimination information flags, for example, a high possibility of residue.

In next step S28, processing for displaying the fluorescence of residue is conducted. Specifically, when the signal data stored in the memory 136 shown in FIG. 14 is read and displayed on the monitor 106, flashing is conducted based on the presence or absence of the flag (code) through the use of the determination information written in the memory unit 138c in the timing for the display of the information on the high possibility of residue.

For example, as is indicated by an example of display screen of the monitor 106 shown in FIG. 12, in the case where a normal image is displayed on a normal image display area 106a and a fluorescence image is displayed on a fluorescence image display area 106b, a fluorescence figure Ia of a lesion site in the fluorescence image is displayed without flashing, whereas a portion of a fluorescence figure Ib which has been determined as being originated from a residue with a high possibility is displayed while flashing.

Therefore, when the portion of fluorescence figure of the residue is displayed, this portion is displayed while flashing and is displayed in a display form different from that of the fluorescence figure of the lesion site. By changing the display form as described above, the user can simply notice the fluorescence portion originated from a residue with a high possibility, based on the resulting fluorescence image.

On the other hand, when the condition Kba<B/A<Hba is not satisfied or the condition Kca<C/A<Hca is not satisfied in the determination processing of steps S25 and S26, step S29 is executed, it is determined that there is a low possibility of fluorescence of residue, and this processing is finished.

In the above description, the determination result of step S29 is not reflected to the display. However, the determination result of step S29 may also be reflected to the display. For example, in the case of step S27, since the possibility of residue is high, the display may be conducted with a high frequency of flashing. The display of the determination result of step S29 may be executed with a low frequency of flashing.

In place of the parallel processing of steps S25 and S26 shown in FIG. 16A, as shown in FIG. 16B, the determination processing of step S25 may be executed, the determination processing of step S26 may further be executed when the condition Kba<B/A<Hba is satisfied, step S27 may further be executed when the condition Kca<C/A<Hca is satisfied, and the signal data in this case may be determined that there is a high possibility of fluorescence of residue.

In this case, the reliability of the result of the determination by step S27 becomes higher. That is, the signal data image-captured through the fluorescence filter 125a and stored into the memory 136 is the fluorescence of residue with a very high possibility.

As described above, according to the present embodiment, even in the case where a fluorescence signal is detected actually, the fluorescence of residue can be effectively discriminated from the fluorescence of lesion site based on the spectrum distribution characteristics and can be displayed in a display form different from that of the lesion site. Therefore, the surgeon can easily grasp the possibility of residue, and can diagnose efficiently.

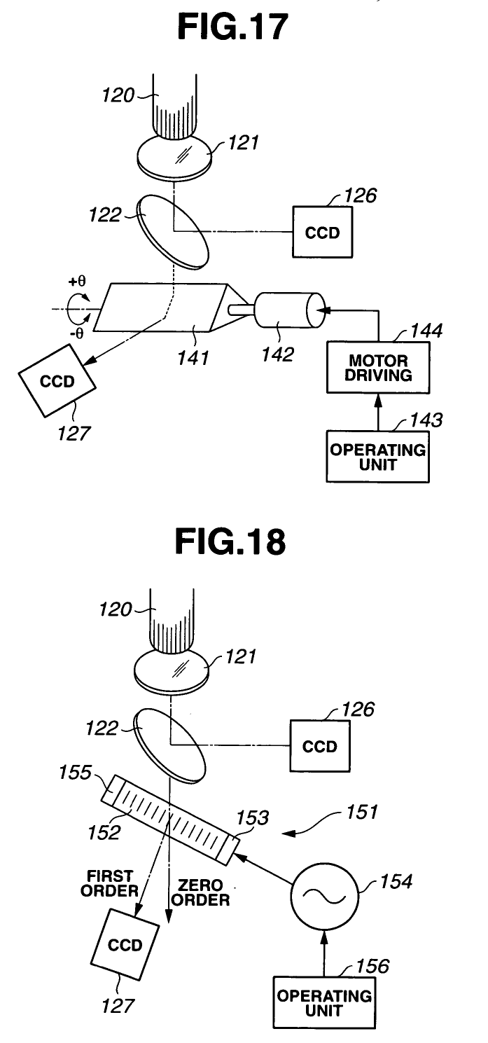

A first modified example of the present embodiment will be described below with reference to FIG. 17. In the second embodiment, a plurality of fluorescence filters 125a, 125b, and 125c are adopted in order to discriminate (determine) the fluorescence figure of residue, the image being mixed into the image pickup of the fluorescence of lesion site.

In the case where the fluorescence spectrum of a residue varies in correspondence to the inspection site in the body, it is predicted that the discrimination can be conducted more reliably by changing the transmission wavelength of the fluorescence filter in correspondence to the variation.

For this purpose, in one method, the number of the fluorescence filters 125a, 125b, and 125c shown in FIG. 12 may be increased, or the rotary plate 124 may be changed and other fluorescence filters may be adopted.

In another method, a spectral prism 141 may be adopted as in the configuration of the first modified example shown in FIG. 17.

That is, as shown in FIG. 17, the spectral prism 141 rotated by a motor 142 is disposed on an optical axis of light passed through the dichroic mirror 122, and the CCD 127 is disposed to receive the light (specifically, fluorescence) dispersed by this spectral prism 141.

This spectral prism 141 is rotated to and fro by the motor 142 within a predetermined angle (for example, from the state at standard angle to +θ and −θ). Since this spectral prism 141 is rotated, the fluorescence dispersed by this spectral prism 141 is continuously varied within a predetermined wavelength band from a wavelength in the state at a standard angle, at which no rotation is conducted, toward the long wavelength side and the short wavelength side, and light can be received (image-captured) by the CCD 127.

According to the present modified example, even in the case where, for example, the wavelength band of a residue fluorescence spectrum is distributed relatively broadly and continuously, as shown in, for example, FIG. 13B, since the wavelength of light to be received (image-captured) can be changed continuously, an effective response can be made. Alternatively, the light may be received substantially with a plurality of discrete wavelengths.

For example, the motor 142 may be composed of a stepping motor, and the rotation angle of the motor 142 may be controlled variably and stepwise by a motor driving circuit 144 through an operating unit 143. That is, the rotation of the motor 142 may be controlled through the motor driving circuit 144, based on the control instruction setting from the operating unit 143 in such a way that the emission time of the fluorescence with a wavelength required to be received by the CCD 127 toward the CCD 127 side is increased and the emission time of the fluorescence with a wavelength not required to be received by the CCD 127 toward the CCD 127 side is decreased.

In this case, the fluorescence wavelength to be substantially received by the CCD 127 can be selected by the selection operation or the like from the operating unit 143, and a response can be made even in the case where the fluorescence spectrum of residue is varied in correspondence to the inspection sites. A response can be made even in the case where the fluorescent drug (fluorescent coloring drug) administered to a lesion site in order to emit fluorescence is changed.

By using the present modified example, a fluorescence spectrum of a previously known residue may be measured, a fluorescence spectrum of a portion containing no residue and provided with a fluorescent drug may be measured, and the fluorescence spectrum used in processing for discriminating the fluorescence of lesion site and the fluorescence of residue in the actual endoscopy may be determined based on these fluorescence spectra attained beforehand.

By using the present modified example, fluorescence spectra of known residue and fluorescent drug may be measured beforehand to attain data of intrinsic fluorescence spectra exhibiting high intensity, and the filter transmission wavelengths of the fluorescence filters 125a, 125b, and 125c shown in FIG. 12 may be set based on the attained data of the fluorescence spectra.

Alternatively, for example, an acoustooptic tunable filter 151 serving as a variable filter may be adopted as in the configuration of the second modified example shown in FIG. 18.

That is, as shown in FIG. 18, the acoustooptic tunable filter (abbreviated as AOTF) 151 composed of tellurium dioxide or the like is disposed on an optical axis of light passed through the dichroic mirror 122, and the CCD 127 is disposed in the direction for receiving the first-order fluorescence diffracted by this AOTF 151.

This AOTF 151 has a configuration in which an RF signal with a variable frequency is applied from a variable RF oscillator 154 to a tellurium dioxide crystal 152 and an acoustic transducer (ultrasonic vibrator) 153 attached to one end surface thereof. The end surface opposite to the end surface provided with the acoustic transducer 153 fixes the tellurium dioxide crystal 152 to a cabinet, although not shown in the drawing, with an absorber 155 to absorb acoustic oscillation therebetween.

By conducting an instruction operation for setting the oscillation frequency of the variable RF oscillator 154 with the operating unit 156, the variable RF oscillator 154 applies the RF signal with the instructed oscillation frequency to the acoustic transducer 153, vibrate the tellurium dioxide crystal 152 with the frequency of the RF signal, and expands and contracts the crystal lattice thereof (refractive index is changed).

Consequently, when fluorescence is incident on this tellurium dioxide crystal 152, a function of such as a transmission grating (diffraction grating) or an optical deflector by Bragg diffraction is performed. In this case, in contrast to usual gratings, this AOTF 151 has a function of diffracting only one specific wavelength, and can realize very narrow-band filter transmission characteristics.

According to the present modified example, fluorescence signal data having nearly ideal filter characteristics and a pass band with a desired wavelength can be attained. consequently, as in the first modified example, a response can be made with flexibility in the case where a fluorescence spectrum of an inspection object site in a body cavity is varied by a residue and in the case where the fluorescence spectrum is varied by a fluorescent drug to be used, and even in such a case, discrimination between the fluorescence figures of the lesion site and the residue can be conducted efficiently.

In the present modified example as well, as described in the first modified example, fluorescence spectra emitted from a residue and a fluorescent agent may be measured beforehand, and the measurement results thereof may be used for setting a fluorescence spectrum used in discriminating the two. In this case, according to the present modified example, selective setting at fluorescence spectra (intrinsic to the residue and the fluorescent drug) effective for discriminating the two can easily be conducted by selectively setting the oscillation frequency, and a plurality of fluorescence spectra can freely be set at their respective fluorescence spectra in a short time since the fluorescence spectra can be changed discretely.

Furthermore, the present modified example has a merit that a mechanical movable portion, which can be moved more macroscopically as compared with that in the first modified example, is not necessary.

### Third Embodiment

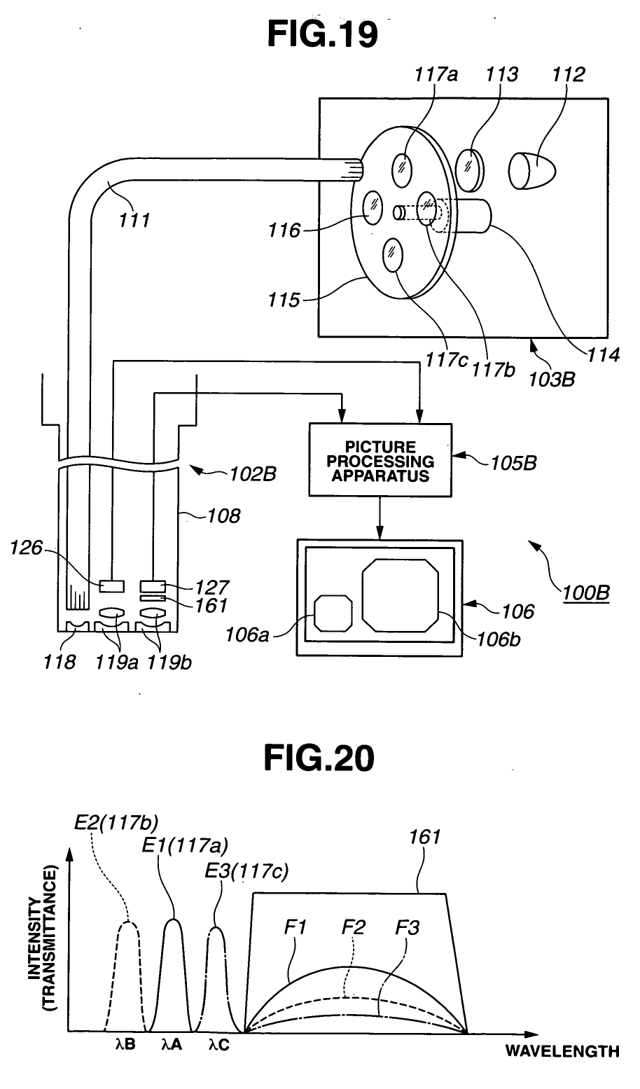

The third embodiment of the present invention will be described below with reference to FIG. 19 to FIG. 21. The same constituents as in the second embodiment are indicated by the same reference numerals as those set forth above and explanations thereof will not be provided. FIG. 19 shows the configuration of a fluorescence observation endoscope apparatus 100B according to the third embodiment of the present invention.

The fluorescence observation endoscope apparatus 100B shown in FIG. 19 comprises an electronic endoscope 102B to be inserted into a body cavity, a light source 103B to supply excitation light and illumination light for normal observation to a light guide 111 of this electronic endoscope 102B, an image processing apparatus 105B to conduct image processing of image pickup signal image-captured by the electronic endoscope 102B, and a monitor 106 to display an image processed by this image processing apparatus 105B.

The electronic endoscope 102B includes a slender insertion portion 108 to be inserted into the body cavity. The light guide 111 is inserted into this insertion portion 108.

The rear end of this light guide 111 is removably connected to the light source 103B. This light source 103B is provided with three excitation light filters 117a, 117b, and 117c as an excitation light irradiation unit to radiate excitation light in a plurality of (three in the drawing) wavelength bands, in place of one excitation light filter 117 disposed on the rotary plate 115 in the light source 103 of the second embodiment.

FIG. 20 shows the spectrum intensity distribution and the like of excitation light passed through the excitation light filters 117a, 117b, and 117c. The excitation light filter 117a generates excitation light El with a wavelength of λA (=λa), which generates fluorescence exhibiting a highest intensity in a fluorescence spectrum distribution of a lesion site. The excitation light filters 117b and 117c transmit excitation light E2 with a wavelength of λB on the side of wavelength shorter than the wavelength of λA and excitation light E3 with a wavelength of λC on the side of wavelength longer than the wavelength of λA (the corresponding excitation light filters 117a, 117b, and 117c are also shown in parentheses in FIG. 20).

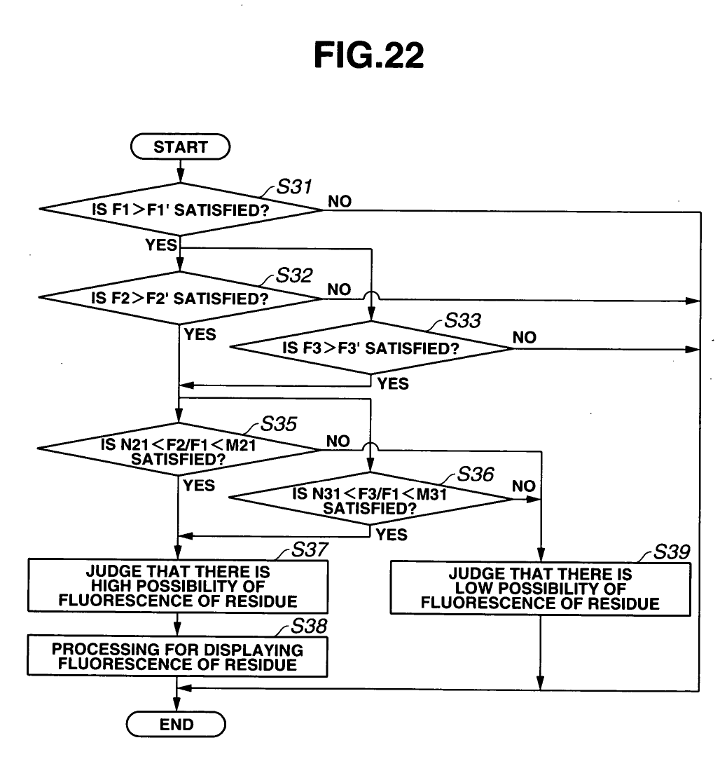

The case where the excitation light filter 117a is set to generate excitation light E1, which generates fluorescence exhibiting a highest intensity in a fluorescence spectrum distribution of a residue, will be described below in the description for FIG. 22.

Since the above-described rotary plate 115 is driven to rotate by a motor 114, white light and the excitation light E1, E2, and E3 sequentially passed through the excitation light filters 117a, 117b, and 117c are incident sequentially on an incident end surface at the rear end of the light guide 111.

The light incident on the incident end surface of the light guide 111 is transmitted to the front-end surface thereof, and is radiated to the side of a subject, e.g., a lesion site or the like in the body cavity 107, from the front-end surface through an illumination lens 118 attached to an illumination window while being diverged.

Two observation windows are disposed adjacent to the illumination window at the distal end portion of the insertion portion 108. Objective lenses 119a and 119b are attached to the two observation windows, and at their respective image generation positions, a CCD 126 for normal observation and a CCD 127 for fluorescence observation are disposed, respectively.

A fluorescence filter 161 to transmit fluorescence in one band is disposed before the CCD 127 for fluorescence observation, serving as a fluorescence image pickup unit. This fluorescence filter 161 has a band-pass filter characteristic of transmitting fluorescence as shown in FIG. 20.

In FIG. 19, signal data attained by image pickup with the CCD 127 based on the excitation light sequentially passed through the excitation light filters 117a, 117b, and 117c are indicated by F1, F2, and F3.

The image pickup signals image-captured with the CCDs 126 and 127 are input into the image processing apparatus 105B, and are subjected to image processing. The resulting image signals are output to the monitor 106, and a normal image and a fluorescence image are displayed on a display surface of the monitor 106.

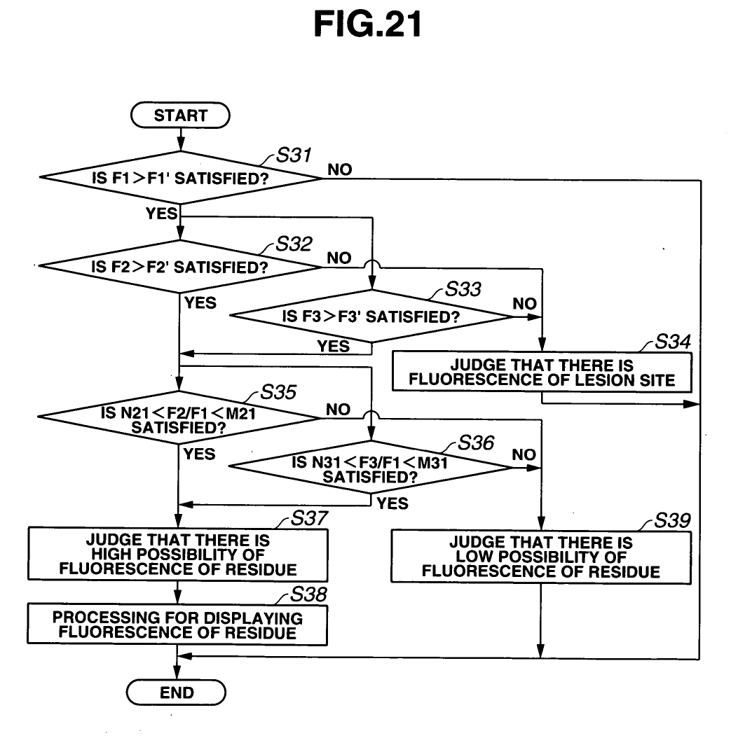

The internal configuration of the image processing apparatus 105B is substantially similar to the configuration of the image processing apparatus 105 in the second embodiment shown in FIG. 14 and, therefore, the operation thereof will be described by using the same reference numerals. However, threshold values stored in the ROM 138b in the CPU 137 are different values from the threshold values in the second embodiment. A residue determination processing substantially similar to that in the second embodiment is conducted by using the threshold values stored in the ROM 138b (described below with reference to FIG. 21).

The operation according to the present embodiment will be described below. The entire actions in the present embodiment are the actions following the flow chart shown in FIG. 15 in the second embodiment.

In the second embodiment, a plurality of fluorescence signal data are attained by using a plurality of fluorescence filters 125a, 125b, and 125c having different transmission bands, the CPU 137 determines whether the plurality of fluorescence signal data are fluorescence signals originated from a lesion site or from a residue. However, in the present embodiment, a plurality of fluorescence signal data are attained by the excitation light E1, E2, and E3 passed through the plurality of excitation light filters 117a, 117b, and 117c sequentially, and the CPU 137 serving as a residue detection unit determines whether the plurality of fluorescence signal data are fluorescence signals originated from a lesion site or from a residue.

Consequently, for the processing of measuring a fluorescence signal spectrum of step S14 shown in FIG. 15, more specifically, the fluorescence signal is measured by changing the irradiation spectrum of the excitation light. After the fluorescence signal is thus measured by changing the irradiation spectrum of the excitation light, the signal data is subjected to the processing of step S15 and the following steps shown in FIG. 15. In more detail, the processing of step S15 and the following steps are the processing shown in FIG. 21.

The determination processing of a residue shown in FIG. 21 will be described. In the present embodiment, F1′, F2′, and F3′ are stored in the ROM 138b in the image processing apparatus 105B as threshold values corresponding to the threshold values Ao, Bo, and Co in the second embodiment. Furthermore, N21, M21, N31 and M31 are stored as threshold values corresponding to the threshold values Kba, Hba, Kca, and Hca in the second embodiment.

That is, when the determination processing of a residue is started, the determination unit 138a of the CPU 137 conducts comparison determination whether the values of the image-captured fluorescence signal data F1, F2, and F3 exceed the threshold values F1′, F2′, and F3′ or not, as indicated by steps S31, S32, and S33 shown in FIG. 21.

In step S31, when the fluorescence signal data F1 is larger than the threshold value F1′, the determination unit 138a determines whether the fluorescence signal data F2 is larger than the threshold value F2′ or not and whether the fluorescence signal data F3 is larger than the threshold value F3′ or not, as indicated by steps S32 and S33. Conversely, when fluorescence signal data A larger than the threshold value F1′ is not detected in step S31, it is determined that there is almost no fluorescence of lesion site nor fluorescence of residue, and this processing is finished.

In the determination processing of steps S32 and S33, when the signal data F2 and F3 do not exceed the threshold values F2′ and F3′, respectively, step 34 is executed, wherein the determination unit 138a determines that the signal data F1 in this case is not originated from a residue, but is originated from the lesion site, and this processing is finished.

On the other hand, in the determination processing of steps S32 and S33, when the signal data F2 and F3 exceed the threshold values F2′ and F3′, since the fluorescence which may be originated from a residue is detected, the determination processing of steps S35 and S36 are executed.

In step S35, the determination unit 138a conducts comparison determination whether the signal data ratio F2/F1 is in between the two threshold values N21 and M21 or not, which are the values used for determining the range corresponding to a residue with a high possibility. The threshold value N21 is a lower limit side threshold value and M21 is an upper limit side threshold value.

Likewise, in step S36 as well, the determination unit 138a conducts comparison determination whether the signal data ratio F3/F1 is in between the two threshold values N31 and M31 or not, which are the values used for determining the range corresponding to a residue with a high possibility. The threshold value N31 is a lower limit side threshold value and M31 is an upper limit side threshold value.

When the condition N21<F2/F1<M21 is satisfied or the condition N31<F3/F1<M31 is satisfied in the determination processing of steps S35 and S36, step S37 is executed. In this case, the determination unit 138a determines that there is a high possibility of a residue. As is described for FIG. 14 in the second embodiment, the determination unit 138a writes the discrimination information based on the determination result into the memory unit 138c. This discrimination information flags, for example, a high possibility of residue.

In next step S38, processing for displaying the fluorescence of residue is conducted. Specifically, when the signal data stored in the memory 136 shown in FIG. 14 is read and displayed on the monitor 106, the flashing control of display is conducted based on the presence or absence of the flag (code) through the use of the discrimination information (determination result information) written in the memory unit 138c in the timing for the display of the information on the high possibility of residue, and the display form is changed. The flashing control of display can be conducted by controlling the oscillation of the oscillator 140b based on the information on the determination result of the memory unit 138c and conducting ON/OFF of the analog switch 140a serving as a residue figure processing unit.

Therefore, when the fluorescence figure portion of the residue is displayed, this portion is displayed while flashing in contrast to that of the fluorescence figure of the lesion site. By changing the display forms as described above, the user can simply notice the fluorescence figure portion originated from a residue with a high possibility, based on the resulting fluorescence image.

On the other hand, when the condition N21<F2/F1<M21 is not satisfied or the condition N31<F3/F1<M31 is not satisfied in the determination processing of steps S35 and S36, step S39 is executed, wherein it is determined that there is a low possibility of fluorescence of residue, and this processing is finished.

In the above description, the determination result of step S39 is not reflected to the display. However, the determination result of step S39 may also be reflected to the display, as described in the second embodiment.

In place of the parallel processing of steps S35 and S36 shown in FIG. 21, as in the case shown in FIG. 16B, the determination processing of step S35 may be executed, the determination processing of step S36 may further be executed when the condition N21<F2/F1<M21 is satisfied, step S37 may further be executed when the condition N31<F3/F1<M31 is satisfied, and the signal data in this case may be determined that there is a high possibility of fluorescence of residue.

In this case, the reliability of the result of the determination by step S37 becomes higher.

As described above, according to the present embodiment, even in the case where a fluorescence signal is detected actually, the fluorescence from residue can be effectively discriminated from the fluorescence from lesion site based on the spectrum distribution characteristics and can be displayed in a display form different from that of the lesion site. Therefore, the surgeon can easily grasp the possibility of residue, and can diagnose efficiently.

In the present embodiment, the excitation light E1 is set in the neighborhood of a wavelength of λA (=λa) suitable for most effectively generating the fluorescence of the lesion site. However, the excitation light E1 may be set at a wavelength suitable for most effectively generating the fluorescence of the residue, one of the other excitation light E2 and E3 may be set at a wavelength suitable for most effectively generating the fluorescence of the lesion site, so as to conduct the determination of the residue.

The processing of the determination of the residue in this case is shown in FIG. 22. In this case, the processing is conducted for the determination of only the residue. The processing shown in FIG. 22 is the processing shown in FIG. 21 except that in steps S32 and S33, when the signal data F2 and F3 do not exceed the threshold values F2′ and F3′, respectively, it is determined that the fluorescence is not originated from a residue, and this processing is finished. The other processing contents are the same as those shown in FIG. 21 and, therefore, the explanations thereof are not provided.

In the above description, the example in which the fluorescence figure that may be originated from a residue is displayed while flashing, is explained as the processing for displaying the fluorescence of residue. However, other than this, the display may be conducted with display color different from the display color for the fluorescence figure of lesion site.

In this case, when a fluorescence figure of residue is present in the fluorescence image by controlling the signal input into one channel, e.g., an R signal channel, of the monitor 106 by, for example, the flag code of the memory unit 138c, display is conducted with a color synthesized by green and blue due to a dropout of color signal of R, whereas the fluorescence figure of the lesion site is displayed monochromatically.

A plurality of laser light sources (not shown in the drawing) may be used as the excitation light irradiation unit in place of the lamp 112 and the excitation light filter 117. Here, each laser light source of the plurality of laser light sources is configured to emit excitation light in a mutually different wavelength band, and is controlled to emit light sequentially. In this manner, the plurality of excitation light in different wavelength bands are sequentially incident on the incident end surface of the light guide 111, are transferred by the light guide 111, and are radiated to the side of the subject, e.g., a lesion site, in the body cavity through the illumination lens 118. According to this configuration, since there is no need to dispose the plurality of excitation light filters 117a, 117b, and 117c and the like, the light source 103B can be miniaturized. Similar operations and effects are exerted when LED or the like is used other than the laser light source.

According to the second and third embodiments of the present invention, when the excitation light is radiated so as to conduct fluorescence image pickup, and the fluorescence image resulting from the fluorescence image pickup is displayed on the monitor, the fluorescence figure originated from the residue may be displayed. However, the fluorescence figure originated from the residue can simply be recognized by discriminating the fluorescence figure of the residue, based on the fluorescence signal attained through the use of the filter set at a fluorescence wavelength intrinsic to the residue or the excitation light filter to generate the fluorescence and conducting display in a display form different from that of the fluorescence figure originated from the lesion site.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A fluorescence observation endoscope apparatus comprising:
an endoscope to radiate excitation light from a light source to the inside of a body cavity and pick up a fluorescence figure from a living body tissue in the body cavity; and
a fluorescence image generation unit to conduct signal processing of an image pickup signal of the fluorescence figure and generate a fluorescence image,
wherein the fluorescence image generation unit comprises:
a target region discrimination unit to calculate the fluorescence level of a predetermined region on the fluorescence image, compare the resulting level with a predetermined fluorescence level, and discriminate a target region on the fluorescence image; and
a target region notification unit to notify the presence of the target region.

2. The fluorescence observation endoscope apparatus according to claim 1, wherein the target region notification unit notifies by a sound.

3. The fluorescence observation endoscope apparatus according to claim 1, wherein the target region notification unit notifies by displaying a graphic symbol in the vicinity of the target region on the fluorescence image.

4. The fluorescence observation endoscope apparatus according to claim 3, wherein the target region notification unit changes any one of the luminance, the color, the shape, and the display position of the graphic symbol in correspondence to the target region.

5. The fluorescence observation endoscope apparatus according to claim 1,
wherein the endoscope comprises a spectrum generation unit to generate a fluorescence signal spectrum of the fluorescence;
the target region discrimination unit comprises a residue detection unit to detect a residue present in the body cavity, based on the fluorescence signal spectrum; and
the target region notification unit comprises a residue figure processing unit to change the display form of the fluorescence figure of the residue on the fluorescence image.

6. The fluorescence observation endoscope apparatus according to claim 5, wherein the residue detection unit comprises:
a spectrum comparison unit to compare the fluorescence signal spectrum with an intrinsic fluorescence spectrum of a lesion site in the body cavity or a residue fluorescence spectrum;
a residue determination unit to determine the presence or absence of the residue based on the information attained in the spectrum comparison unit; and
a residue position detection unit to detect the position and the size of the residue.

7. The fluorescence observation endoscope apparatus according to claim 5, wherein the residue figure processing unit displays the fluorescence figure of the residue on the image display unit, as distinguished from other fluorescence figures.

8. The fluorescence observation endoscope apparatus according to claim 5, wherein the light source comprises an excitation light irradiation unit to radiate excitation light in one band to the inside of the body cavity, the spectrum generation unit has a plurality of band-pass filters to cut the excitation light and transmit the fluorescence in a plurality of bands, and the fluorescence signal spectrum is generated based on the fluorescence passed through the plurality of band-pass filters.

9. The fluorescence observation endoscope apparatus according to claim 1,
wherein the light source comprises an excitation light irradiation unit to radiate excitation light in a plurality of bands to the inside of the body cavity;
the endoscope comprises a filter to transmit fluorescence in one band and a fluorescence image pickup unit to pick up the fluorescence figure passed through the filter;
the target region discrimination unit comprises a residue detection unit to detect a residue present in the body cavity based on the signal of the fluorescence image attained by the fluorescence image pickup unit; and
the target region notification unit comprises a residue figure processing unit to change the display form of the fluorescence figure of the residue on the fluorescence image.

10. A fluorescence observation method comprising:
an image pickup step of picking up an image of fluorescence from a living body tissue in a body cavity by radiating excitation light to the inside of the body cavity;
a fluorescence image generation step of conducting signal processing of an image pickup signal of the fluorescence figure and generating a fluorescence image;
a target region extraction step of calculating the fluorescence level of a predetermined region on the fluorescence image, comparing the resulting level with a predetermined fluorescence level, and extracting a target region on the fluorescence image; and
a target region notification step of notifying the presence of the target region.

11. The fluorescence observation method according to claim 10,
wherein the image pickup step comprises a spectrum generation step of generating a fluorescence signal spectrum of the fluorescence;
the target region extraction step comprises a residue detection step of detecting a residue present in the body cavity, based on the fluorescence signal spectrum; and
the target region notification step comprises a residue figure processing step of changing the display form of the fluorescence figure of the residue on the fluorescence image.

12. The fluorescence observation method according to claim 11, wherein the residue detection step comprises:
a spectrum comparison step of comparing the fluorescence signal spectrum with an intrinsic fluorescence spectrum of a lesion site in the body cavity or an intrinsic fluorescence spectrum of a residue;
a residue determination step of determining the presence or absence of the residue based on the information attained in the spectrum comparison step; and
a position detection step of detecting the position and the size of the residue.

13. The fluorescence observation method according to claim 11, wherein the residue figure processing step comprises a step of displaying the fluorescence figure of the residue, as distinguished from other fluorescence figures.
